(19) United States
(12) Patent Application Publication
(10) Pub. No.: US 2020/0077992 A1
(43) Pub. Date: Mar. 12, 2020

(54) Device for Collecting a Biological Sample

(71) Applicant: Case Western Reserve University, Cleveland, OH (US)

(72) Inventors: Sanford Markowitz, Cleveland, OH (US); Dean Secrest, Cleveland, OH (US); Amitabh Chak, Cleveland, OH (US); Joseph Willis, Cleveland, OH (US); Dennis Siedlak, Cleveland, OH (US)

(21) Appl. No.: 16/610,115

(22) PCT Filed: May 3, 2018
(86) PCT No.: PCT/US2018/030907

§ 371 Date: Nov. 1, 2019

Related U.S. Application Data
(60) Provisional application No. 62/500,933, filed on May 3, 2017.

(51) Int. Cl. A61B 10/04

(52) U.S. Cl. CPC A61B 10/04; A61B 2010/0216

(57) ABSTRACT

A device for collecting a biological sample in an esophagus of a patient includes a swallowable collection portion for collecting a sample at a collection site in the esophagus. A stylet is connected with the collection portion for placing the collection portion into the back of a throat of a patient for swallowing. The device may have a collection portion having a first axial end portion and a second axial end portion. A sleeve is in the first axial end portion. The device may have at least one tissue collecting projection extending from an outer surface of the collection portion; a first side wall of the tissue collecting; a second wall of the tissue collecting projection. A method for collecting a biological sample includes moving the swallowable collection portion with the stylet into the back of a throat of a patient for swallowing.

### RELATED APPLICATIONS

This application claims priority from U.S. Provisional Patent Application Ser. No. 62/500,933 filed May 3, 2017, the subject matter of which is incorporated herein by reference in its entirety.

### GOVERNMENT FUNDING

This invention was made with government support under Grant Nos. P50CA150964, U01CA152756, U54CA163060 awarded by The National Institutes of Health. The United States government has certain rights to the invention.

### BACKGROUND OF THE INVENTION

The present invention is directed to a device for collecting a biological sample, and more specifically, to a device for collecting a biological sample, such as tissue, cells, protein, RNA and/or DNA from an esophagus of a patient.

A known tissue collection device includes an expandable device with longitudinally extending folds. The expandable device expands radially at a collection site within a body lumen, such as an esophagus. After the device is expanded, tissue is collected from the collection site. The expandable device is deflated after tissue is collected. The folds trap collected tissue when the device is deflated after collection of the tissue. The known tissue collection device may be inserted through an endoscope to the collection site or via standard catheter intubation techniques.

### SUMMARY OF THE INVENTION

The present invention relates to a device for collecting a biological sample in an esophagus of a patient. The device includes a swallowable collection portion for collecting a sample at a collection site in the esophagus. A stylet connected with the collection portion helps place the collection portion into the back of a throat of a patient for swallowing.

In another aspect of the present invention, a device for collecting a biological sample in an esophagus of a patient includes a collection portion having a first axial end portion and a second axial end portion. The second axial end portion has a collapsed position and an expanded position. The second axial end portion moves in an axial direction relative to the first axial end portion when the second axial end portion moves between the collapsed position and the expanded position. The second axial end portion extends axially into the first axial end portion and has a concave shape when the second axial end portion is in the collapsed position. A sleeve is in the first axial end portion.

In another aspect of the present invention, a device for collecting a biological sample in an esophagus of a patient includes a collection portion having a collapsed position and an expanded position. At least one tissue collecting projection extends from an outer surface of the collection portion. A first side wall of the tissue collecting projection extends generally perpendicular to the outer surface of the collection portion when the collection portion is in a non-inflated position between the collapsed and expanded positions. A second wall of the tissue collecting projection tapers toward the first side wall as the side walls extend radially outward from the outer surface when the collection portion is in the non-inflated position between the collapsed and expanded positions.

In another aspect of the present invention, a method for collecting a biological sample includes moving a swallowable collection portion with a stylet into the back of a throat of a patient for swallowing. The collection portion is moved to a collection site in the esophagus with a portion of the collection portion in a collapsed position. The portion of the collection portion is expanded when the collection portion is at the collection site. A biological sample is collected with the portion of the collection portion in the expanded position. The portion of the collection portion is collapsed after collecting the sample. The device is removed from the esophagus of the patient

### BRIEF DESCRIPTION OF THE DRAWINGS

The foregoing and other features of the present invention will become apparent to those skilled in the art to which the present invention relates upon reading the following description with reference to the accompanying drawings, in which:

FIG. 1 is a schematic pictorial view of a biological sample collection device constructed in accordance with the present invention;

FIG. 2 is a schematic pictorial view of the collection device of FIG. 1 shown in a collapsed position;

FIG. 3 is a sectional view of the collection device of FIG. 2;

FIG. 4 is an enlarged plan view of a projection or bristle of the collection device of FIG. 1;

FIG. 5 is a sectional view of the projection taken along the line 5-5 in FIG. 4;

FIG. 6 is an enlarged plan view of the projection shown after expansion of a portion of the collection device;

FIG. 7 is a schematic view of the projection after expansion of the portion of the collection device taken along the line 7-7 in FIG. 6;

FIG. 8 is a schematic pictorial view of the collection device showing a stylet and connector of the collection device;

FIG. 9 is an enlarge schematic pictorial view of the connector of FIG. 8;

FIG. 10 is a schematic sectional view of a collection device constructed in accordance with another embodiment of the present invention;

FIG. 11 is a schematic pictorial view of a collection device constructed in accordance with a third embodiment of the present invention;

FIG. 12 is a schematic sectional view of a collection device constructed in accordance with a fourth embodiment of the present invention; and

FIG. 13 is a schematic sectional view of a collection device constructed in accordance with fifth embodiment of the present invention.

### DESCRIPTION OF THE INVENTION

A collection device 10 for collecting a biological sample constructed in accordance with the present invention is illustrated in FIGS. 1-9. The collection device 10 may be used to collect tissue, cells, protein, RNA and/or DNA from a body lumen, such as an esophagus of a patient. The tissue, cells, protein, RNA and/or DNA collected from the esophagus may be used in any one of the methods disclosed in U.S. patent application Ser. No.14/109,041, U.S. patent application Ser. No.13/670,155, U.S. patent application Ser. No. 13/263,020, U.S. Pat. No. 8,642,271, U.S. Pat. No. 8,481,707, U.S. Pat. No. 8,415,100, U.S. Patent No. 8,221,977, U.S. Pat. No. 7,964,353, and U.S. Pat. No. 7,485,420, which are incorporated herein by reference in their entirety.

The collection device 10 includes a generally hollow longitudinally extending collection portion 12. The collection portion 12 has a first or proximal axial end portion 14 connected to a second or distal axial end portion 16. The distal end portion 16 has a first axial end portion 22 connected to the proximal axial end portion 14. The first end portion 22 may be connected to the proximal end portion 14 in any desired manner, such as by using an adhesive or bonding. The first axial end portion 22 engages a shoulder 24 on the proximal axial end portion 14. Therefore, the collection portion 12 has a smooth outer surface. The distal axial end portion 16 may be connected to the proximal end portion 14 in any desired manner. The proximal axial end portion 14 and the distal axial end portion 16 may be made of a flexible polymer, such as silicone or polyurethane. The distal axial end portion 16 has a lower durometer than the proximal axial end portion 14. The distal axial end portion 16 may have a durometer between 5-90 Shore A. The durometer of the distal axial end portion 16 is preferably between 20-70 Shore A, and more specifically, approximately 30 Shore A.

The distal axial end portion 16 may expand and contract. The first or proximal axial end portion 14 is relatively rigid. Therefore, the proximal end portion 14 has a fixed radial extent. The first axial end portion 14 and the second axial end portion 16 may be formed as separate pieces that are connected together in any desire manner or may be integrally formed as one-piece. Although the proximal end portion 14 is illustrated as having a cylindrical shape, the proximal end portion may have any desired shape.

The proximal axial end portion 14 is connected to a support member 20, such as a catheter. The support member 20 may be a tubular member in fluid communication with the interior of the collection portion 12. The proximal axial end portion 14 conducts fluid, such as air, from the support member 20 to the distal axial end portion 16. The support 20 resists collapsing when a vacuum is applied to the support member and resists stretching during withdrawal of the collection device 10 from the collection site.

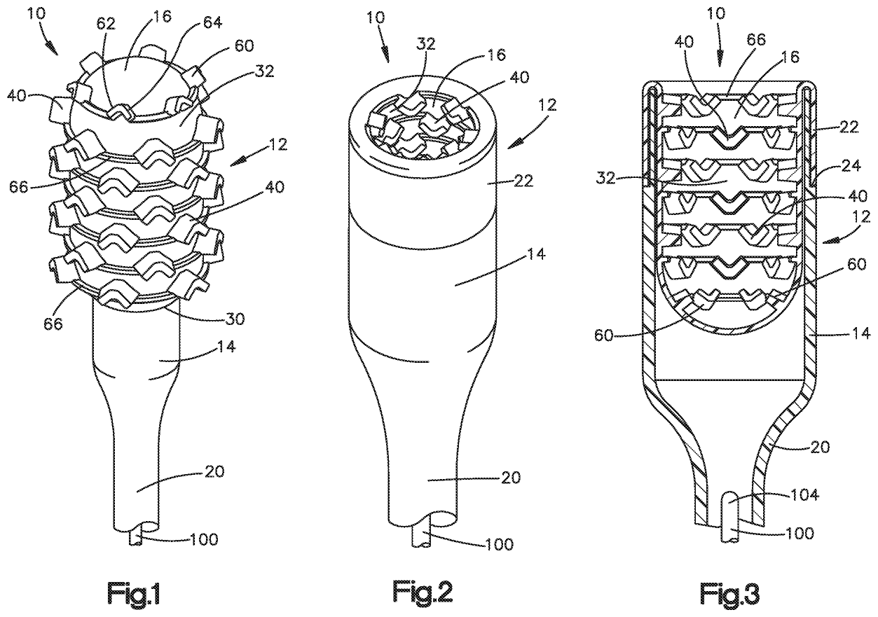

The second or distal end portion 16 of the collection portion 12 has an expanded or inflated position (FIG. 1) and a collapsed or deflated position (FIGS. 2-3). The expanded position shown in FIG. 1 may be one of many expanded positions for the distal end portion 16. It is contemplated that the distal end portion 16 may expand more than shown in FIG. 1 so that the distal end portion obtains a more spherical shape and looks similar to a hot air balloon. The distal end portion 16 has a convex shape, shown in FIG. 1, when in the expanded or inflated position. The distal end portion 16 may extend radially outward a greater distance than the proximal end portion 14 when in the expanded position.

The distal end portion 16 extends into the first or proximal axial end portion 14 and has a concave shape, shown in FIGS. 2 and 3, when in the collapsed or deflated position. The distal end portion 16 may be inverted when in the collapsed position. The distal end portion 16 extends axially into the interior of the proximal end portion 14 when in the collapsed or deflated position. Therefore, the distal end portion 16 moves axially or longitudinally relative to the proximal end portion 14 when moving between the deflated and inflated positions. The relatively lower durometer of the distal end portion 16 allows the distal end portion to extend axially into the interior of the proximal end portion 14 and have a concave shape when in the collapsed position. The distal end portion 16 may be biased into the collapsed or deflated position in any desired manner

The proximal end portion 14 has a relatively high durometer so that the proximal end portion does not collapse when a vacuum is applied to the proximal end portion through the support 20. The shape of the proximal end portion 14 does not change when the distal end portion 16 moves between the deflated and inflated positions. The proximal end portion 14 does not move radially when the distal end portion 16 moves between the deflated and inflated positions.

The distal end portion 16 has an outer surface 32 for collecting tissue when the distal portion is in the expanded position. The outer surface 32 faces radially outwardly when the distal end portion 16 is in the expanded position and may face radially inwardly when the distal end portion is in the collapsed or inverted position. It is contemplated that the outer surface 32 of the distal end portion 16 may have any desired construction for collecting tissue. The outer surface 32 of the distal end portion 16 may have a plurality of projections or bristles 40 for collecting tissue. The distal end portion 16 may have any desired number of projections or bristles 40.

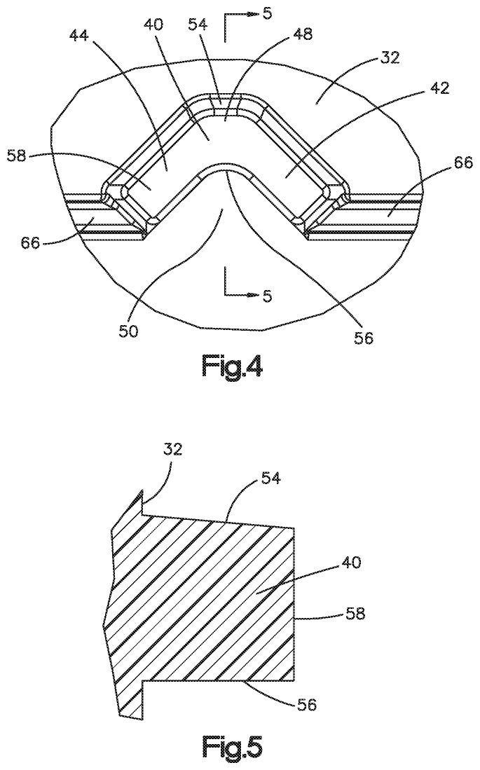

The projections or bristles 40 may have a V-shape (FIG. 4). Each projection 40 has a first side 42 and a second side 44 extending from an intersection 48. The first and second sides 42, 44 extend in a generally proximal direction from the intersection 48 when the distal end portion 16 is in the expanded position (FIG. 1). The first and second sides 42, 44 extend in a generally distal direction when the distal end portion 16 is in the collapsed or inverted position (FIGS. 2 and 3). The first and second sides 42, 44 define a cup 50 for receiving collected biological samples. The cup 50 faces in a proximal direction when the distal portion 16 is in the expanded position and faces in a distal direction when the distal portion is in the collapsed position.

The first and second sides 42, 44 may extend at an angle of approximately 90° relative to each other. It is contemplated that the first and second sides 42 and 44 may extend at any desired angle relative to each other. The desired angle may be determined based on the type of biological sample to be collected. Alternatively, the projections 40 may be cup shaped or have a semi-circular shape.

Each of the projections or bristles 40 has side walls 54 and 56 (FIG. 5) that extend radially outward from the outer surface 32 when the distal portion 16 is in the expanded position. The side wall 56 faces the proximal direction when the distal portion is in the expanded position and forms an inner side of the cup 50. The side wall 54 faces the distal direction when the distal portion is in the expanded position and forms an outer wall of the cup 50. The side walls 54 and 56 extend from the outer surface 32 to a radially outer surface 58 of the projection 40. The side wall 56 extends generally perpendicular to the outer surface 32 and the outer surface 58 of the projection 40 when the distal end portion 16 is in a non-inflated position between the expanded and collapsed positions. The side wall 54 tapers toward the side wall 56 as the side wall 54 extends from the outer surface 32 toward the radially outer surface 58 of the projection 40 when the distal end portion 16 is in the non-inflated position.

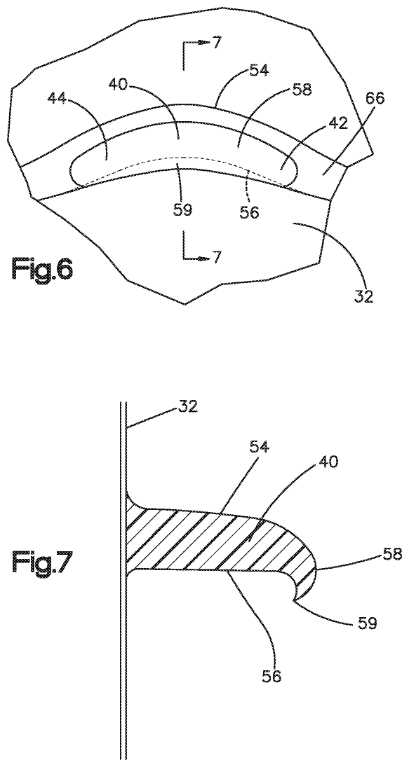

The side wall 56 may form a flap, hood or lip 59 (FIGS. 6-7) when the distal end portion 16 is in the expanded position. The lip 59 helps collect a sample for the collection site. The lip 59 extends from the outer surface 58 of the projection 40 toward the proximal end portion 14. The projection 40 elongates from the shape shown in FIG. 4 to the shape shown in FIG. 6 during expansion of the distal end portion 16. The projection 40 also reduces height from the shape shown in FIG. 5 to the shape shown in FIG. 7 during expansion of the distal end portion 16. The elongation and reduction in height of the projection 40 causes the collection lip 59 to form on the collection side of the projection 40. The difference in the tapers between the side walls 54, 56 creates a bias lean of the wall section to roll toward the side wall 56. The projection 40 is biased and concave on the side wall 56 in the non-inflated state and this is further enhanced during inflation. When the elongation of the projection 40 occurs, the projection thins out and becomes less stable to remain in a vertical column which causes the top edge to roll over towards the side wall 56 which forms the lip 59 over the proximal or collection side of the projection. The side walls 54, 56 may both taper at any desired angles. It is also contemplated that the side walls 54 and 56 may not taper toward each other.

The distal end portion 16 may include a plurality of projections or bristles 60 (FIG. 1) extending from a distal portion of the distal end portion 16. The projections 60 have the same general V-shape as the projections 40 and are smaller than the projections 40. The projections 60 have first and second sides 62 and 64 that have a length smaller than the first and second sides 42, 44 of the projections 40.

The projections or bristles 40, 60 are arranged in circumferentially extending rows (FIG. 1). It is contemplated that each row has six projections 40 or 60. It is contemplated that each of the rows may have any desired number of projections 40 or 60. Each of the projections 40, 60 is circumferentially offset from the projections on an adjacent row. Ribs 66 extend circumferentially between adjacent projections 40, 60 in each row. The ribs 66 extend between ends of the sides walls 54, 56 opposite the intersections 48.

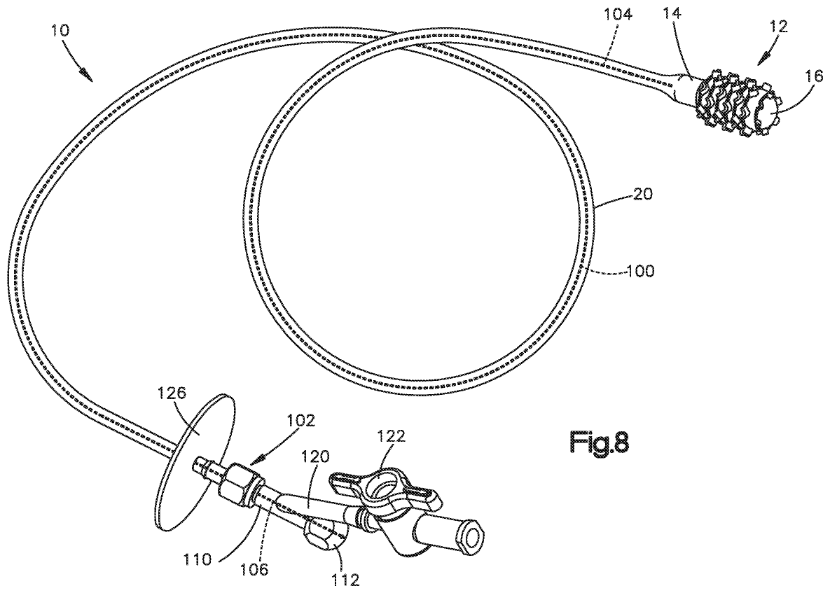

The catheter 20 may have a stylet 100 (FIG. 8) that provides stiffness to the catheter 20 so that a physician or operator may place the collection portion 12 into the back of a throat of a patient for easier swallowing. The stylet 100 may extend through the catheter 20 from adjacent the first or proximal axial end portion 14 of the collection portion 12 to a connector 102. The connector 102 is connected with the catheter 20 and permits the introduction of fluid into the catheter for expanding the distal end portion 16 of the collection portion 12. The stylet 100 is preferably made of a polyether ether ketone (PEEK) polymer. However, the stylet may be a stainless steel guidewire, a polymer monofilament extrusion and/or a stainless steel monofilament core wire. The stylet 100 may have a rounded flexible distal end 104 (see FIG. 3) spaced from the collection portion 12. The flexible distal end 104 may be a graduated ground tip for increased flexibility. The distal end 104 may be the most flexible portion of the stylet 100.

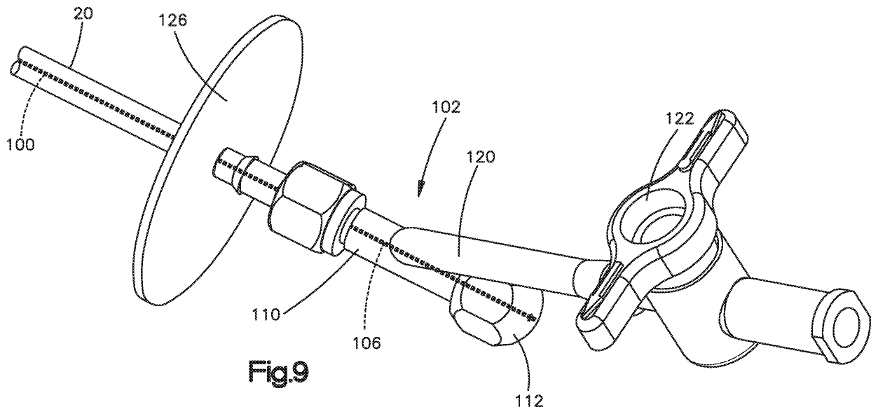

A proximal end 106 (FIGS. 8-9) of the catheter 20 is connected to the connector 102. The connector 102 may be a Y-fitting with a first branch 110 connected to the proximal end 106 of the stylet 100. The proximal end 106 of the stylet 100 extends through the first branch 110 into a cap 112 that seals and closes the first branch. The proximal end 106 is connected to the cap 112 and the first branch 110 with epoxy and cut off flush with the proximal end of cap 112. The epoxy may connect the cap 112 to the first branch 110. It is contemplated that the stylet 100 may be fixedly connected to the cap 112, such as by insert molding. The stylet 100 may then be inserted into the Y-fitting 102 and catheter 20 and connected to the Y-fitting by the cap. The stylet 100 could then be removed from the catheter 20 and Y-fitting 102 if desired. The catheter 20 may be lubricated to permit removal of the stylet 100 from the catheter. It is also contemplated that the proximal end 106 may extend through a Tuohy-Borst adapter connected to the first branch 110 to allow a user to loosen the Tuohy-Borst adapter and remove the stylet 100 to reduce the stiffness of the catheter 20. It is also contemplated that the stylet may extend along the outside of the catheter 20.

The Y-fitting 102 has a second branch 120 extending at an angle to the first branch 110. The second branch 120 may have a stopcock 122 for opening and closing the second branch. A syringe may be connected to the second branch 120 for introducing a fluid, such as air, into the Y-fitting 102 and catheter 20 to expand the distal end portion 16 of the collection portion 12 and apply a vacuum to remove the fluid to collapse the distal end portion 16 after collecting a sample. The stopcock 122 may be used to retain the fluid in the catheter 20 and collection portion 12 when obtaining a sample. The stopcock 122 and syringe help to control the injection of fluid to move the distal end portion 16 between the collapsed and expanded positions.

A disk 126 may be connected to a proximal end of the catheter 20 or the distal end of the connector 102. The disk 126 extends radially away from the catheter 20 to prevent the connector 102 from being inserted into a patient's mouth and/or throat.

The collection portion 12 is moved to a collection site within a body lumen, such as an esophagus, with the distal end portion 16 in the collapsed or deflated position. The collection portion 12 may be swallowed by a patient. The stylet 100 may be manipulated to place the collection portion 12 into the back of the throat of the patient to help with the swallowing of the collection portion. It is also contemplated that the patient may be intubated with the collection portion 12 attached to the catheter. The distal end portion 16 may be held in the collapsed or deflated position by applying a vacuum to the collection portion 12 through the support 20. The support member 20 or catheter may have depth markings to determine the collection site within the patient's anatomy. The collection portion 12 may be moved past a lower esophageal sphincter (LES) and pulled in a proximal direction toward the LES. The operator or physician may sense the increased tension in the catheter 20 when the collection portion 12 engages the LES. The distal end portion 16 of the collection portion 12 may be expanded when the LES is sensed. The distal end portion 16 is moved from the collapsed position to the expanded position when the collection portion 12 is at or near the collection site. The syringe connected to the Y-fitting 102 may be activated to apply pressurized fluid, such as air, to the distal end portion 16 to cause the distal end portion to move axially from the collapsed position to the expanded position.

The collection portion 12 is moved in the esophagus or body lumen to collect a biological sample, such as, tissue, cells, protein, RNA and/or DNA from the collection site when the distal end portion 16 is in the expanded position. It is contemplated that the collection portion 12 is only moved in a proximal direction so that the expanded distal end portion 16 engages the collection site to collect biological samples. The depth markings on the support member 20 or catheter may be used as a guide. After the biological sample is collected, the distal end portion 16 is moved from the expanded position to the collapsed or inverted position. The distal end portion 16 may be moved from the expanded position to the collapsed position by applying a vacuum to the collection portion 12 with the syringe connected to the Y-fitting 102. As the collection portion 12 moves out of the body lumen, the distal end portion 16 does not engage the body lumen and prevents the collected biological samples from being contaminated by tissue from areas along the body lumen different from the collection site. Once the collection device 10 is removed from the patient, the biological samples are collected via a wash and/or the collection portion 12 or the distal end portion 16 may be cut from the support member 20 and deposited in a biological sample vial.

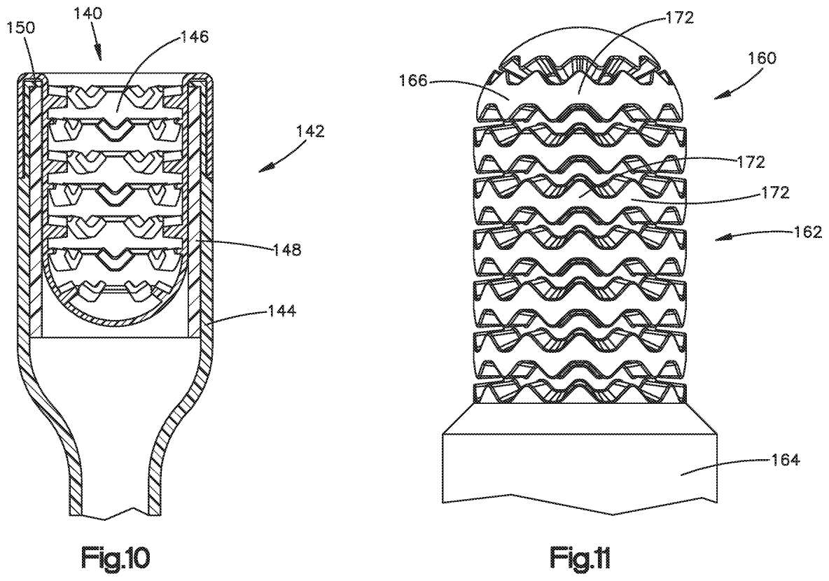

Another embodiment of a collection device 140 is shown in FIG. 10. The collection device 140 is generally similar to the collection device shown in FIGS. 1-9 and has a collection portion 142 with a first or proximal end portion 144 and a second or distal end portion 146. The distal axial end portion 146 may expand and contract. The first or proximal axial end portion 144 is relatively rigid. Therefore, the proximal end portion 144 has a fixed radial extent. The second or distal end portion 146 of the collection portion 142 has an expanded or inflated position, similar to the expanded position shown in FIG. 1, and a collapsed or deflated position shown in FIG. 10. The distal end portion 146 has a convex shape when in the expanded or inflated position. The distal end portion 146 extends into the first or proximal axial end portion 144 and has a concave shape when in the collapsed or deflated position. The distal end portion 146 may be inverted when in the collapsed position. The distal end portion 146 extends axially into the interior of the proximal end portion 144 when in the collapsed or deflated position. Therefore, the distal end portion 146 moves axially or longitudinally relative to the proximal end portion 144 when moving between the deflated and inflated positions.

A stiffening sleeve 148 is connected to the proximal end portion 144. The sleeve 148 may be axially inserted into the proximal end portion 144 of the collection portion 142 so that the distal end portion 146 extends into the sleeve when the distal end portion is in the collapsed position. The sleeve 148 is retained in the proximal end portion 144 by a distal undercut rim 150 on the proximal end portion. The sleeve 148 may be inserted axially into the proximal end portion 144 until the undercut rim 150 snaps over the sleeve to retain the sleeve in the proximal end portion. The undercut rim prevents the sleeve 148 form being able to slide out into the distal end portion 146. The sleeve 148 may be a polypropylene molded cylinder that provides additional column strength to the proximal end portion 144 to help prevent column and side wall collapse during vacuum inversion of the distal end portion 146. The sleeve 148 allows for a thinner wall of the proximal end portion 144. The thinner wall of the proximal end portion 144 provides more space on the inside of the proximal end portion for the distal end portion 146 to invert easier. The ease at which the distal end portion 146 inverts may enhance the ability to collect as much of the biological sample as possible. If there is too much friction between the surfaces of the distal end portion 146 as the distal end portion inverts into the proximal end portion 144 it could squeegee off the sample. The sleeve 148 enhances inversion reliability and reduces the surfaces of the distal end portion 146 from rubbing against each other during the inversion. The sleeve 148 may be a polymer and/or metallic thin wall sleeve inserted or insert molded into the proximal end portion 144. The sleeve 148 provides hoop strength and helps prevent the proximal end portion 144 from collapsing under vacuum.

The distal end portion 146 has an outer surface for collecting tissue when the distal portion is in the expanded position. The outer surface faces radially outwardly when the distal end portion 146 is in the expanded position and may face radially inwardly when the distal end portion is in the collapsed or inverted position. The outer surface of the distal end portion 146 may have a plurality of projections or bristles 152 for collecting tissue. The projections 152 may have a V-shape similar to the V-shaped projections 40 illustrated in FIGS. 1-7.

Another embodiment of a collection device 160 is illustrated in FIG. 11. The collection device 160 is generally similar to the collection device shown in FIGS. 1-9, however, the collection device 160 illustrated in FIG. 11 has a double V texture. The collection device 160 has a collection portion 162 with a first or proximal end portion 164 and a second or distal end portion 166. The distal axial end portion 166 may expand and contract. The first or proximal axial end portion 164 is relatively rigid. Therefore, the proximal end portion 164 has a fixed radial extent. The second or distal end portion 166 of the collection portion 162 has an expanded or inflated position, similar to the expanded position shown in FIG. 1, and a collapsed or deflated position, similar to the collapsed position shown in FIG. 3. The collection portion 162 is shown in FIG. 11 in a non-inflated position between the expanded and collapsed positions. The distal end portion 166 has a convex shape when in the expanded or inflated position. The distal end portion 166 extends into the first or proximal axial end portion 164 and has a concave shape when in the collapsed or deflated position. The distal end portion 166 extends axially into the interior of the proximal end portion 164 when in the collapsed or deflated position. Therefore, the distal end portion 166 moves axially or longitudinally relative to the proximal end portion 164 when moving between the deflated and inflated positions.

The distal end portion 166 has an outer surface for collecting tissue when the distal portion is in the expanded position. The outer surface faces radially outwardly when the distal end portion 166 is in the expanded position and may face radially inwardly when the distal end portion is in the collapsed or inverted position. The outer surface of the distal end portion 166 may have a plurality of projections or bristles 172 for collecting tissue. The projections 172 may form a double V-shape. Each of the projections 172 is similar to the V-shaped projections 40 illustrated in FIGS. 1-7. Each V-shaped projection 172 is connected directly to an adjacent V-shaped projection.

Another embodiment of a collection device 180 is illustrated in FIG. 12. The collection device 180 is generally similar to the collection device shown in FIGS. 1-9 and has a collection portion 182 with a first or proximal end portion 184 and a second or distal end portion 186. The distal axial end portion 186 may expand and contract. The first or proximal axial end portion 184 is relatively rigid. Therefore, the proximal end portion 184 has a fixed radial extent. The second or distal end portion 186 of the collection portion 182 has an expanded or inflated position, similar to the expanded position shown in FIG. 1, and a collapsed or deflated position shown in FIG. 12. The distal end portion 186 has a convex shape when in the expanded or inflated position. The distal end portion 186 extends into the first or proximal axial end portion 184 and has a concave shape when in the collapsed or deflated position. The distal end portion 186 may be inverted when in the collapsed position. The distal end portion 186 extends axially into the interior of the proximal end portion 184 when in the collapsed or deflated position. Therefore, the distal end portion 186 moves axially or longitudinally relative to the proximal end portion 184 when moving between the deflated and inflated position.

The distal end portion 186 has an outer surface for collecting tissue when the distal portion is in the expanded position. The outer surface faces radially outwardly when the distal end portion 186 is in the expanded position and may face radially inwardly when the distal end portion is in the collapsed or inverted position. The outer surface of the distal end portion 186 may have a plurality of projections or bristles 192 for collecting tissue. The projections 192 may have a V-shape similar to the V-shaped projections 40 illustrated in FIGS. 1-7.

The collection device 180 includes a gelcap or gelatin cover or cap 194 that may be loaded over an end of the collection portion 182. The cap 194 holds the distal end portion 186 in the collapsed position during insertion and movement of the collection portion 182 to the collection site. The cap 194 falls off, pops off and/or dissolves when the collection portion 182 reaches the body lumen. The cap 194 may fall off in response to the movement of the distal end portion 186 from the collapsed position to the expanded position.

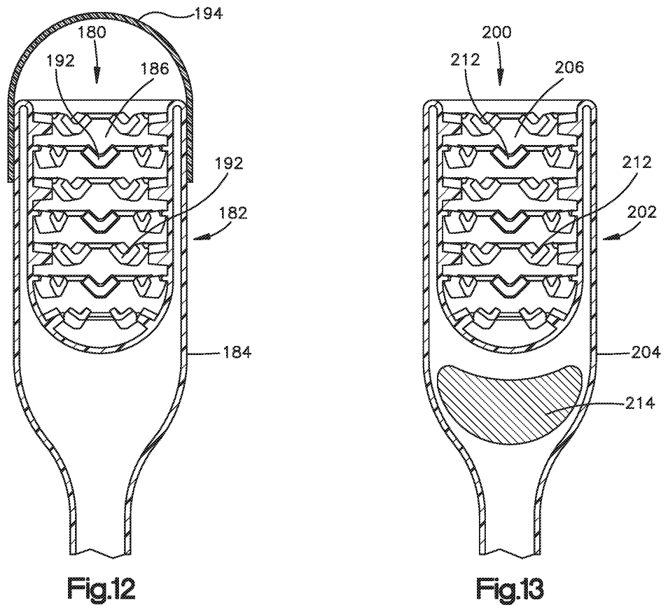

Another embodiment of a collection device 200 is illustrated in FIG. 13. The collection device 200 is generally similar to the collection device shown in FIGS. 1-9 and has a collection portion 202 with a first or proximal end portion 204 and a second or distal end portion 206. The distal axial end portion 206 may expand and contract. The first or proximal axial end portion 204 is relatively rigid. The second or distal end portion 206 of the collection portion 202 has an expanded or inflated position, similar to the expanded position shown in FIG. 1, and a collapsed or deflated position shown in FIG. 13. The distal end portion 206 has a convex shape when in the expanded or inflated position. The distal end portion 206 extends into the first or proximal axial end portion 204 and has a concave shape when in the collapsed or deflated position. The distal end portion 206 may be inverted when in the collapsed position. The distal end portion 206 extends axially into the interior of the proximal end portion 204 when in the collapsed or deflated position. Therefore, the distal end portion 206 moves axially or longitudinally relative to the proximal end portion 204 when moving between the deflated and inflated position.

The distal end portion 206 has an outer surface for collecting tissue when the distal portion is in the expanded position. The outer surface faces radially outwardly when the distal end portion 206 is in the expanded position and may face radially inwardly when the distal end portion is in the collapsed or inverted position. The outer surface of the distal end portion 206 may have a plurality of projections or bristles 212 for collecting tissue. The projections 212 may have a V-shape similar to the V-shaped projections 40 illustrated in FIGS. 1-7.

The collection device 200 includes a weight 214 connected to the proximal end portion 204. The weight 214 may aid in swallowing the collection portion 202. The weight 214 may be made of tungsten and inserted into the proximal end portion 204. It is contemplated that the weight 214 may be insert molded to the proximal end portion 204.

The collection devices 140, 160, 180 and 200 may be used with the catheter 20, stylet 100, and/or connector 102 of FIGS. 8-9. It is also contemplated that the sleeve 148, cap 194 and/or the weight 214 may be used together or separately with any of the collection devices.

From the above description of the invention, those skilled in the art will perceive improvements, changes and modifications. Such improvements, changes and modifications are intended to be covered by the appended claims.

Having described the invention, the following is claimed:

1. A device for collecting a biological sample in an esophagus of a patient, the device comprising:
a swallowable collection portion for collecting a sample at a collection site in the esophagus;
a stylet connected with the collection portion for placing the collection portion into the back of a throat of a patient for swallowing.

2. A device for collecting a biological sample as set forth in claim 1 wherein a catheter is connected to the collection portion and a connector, the stylet extending through the catheter from the stylet toward the collection portion.

3. A device for collecting a biological sample as set forth in claim 2 wherein the connector is a Y-fitting, the stylet being connected to a first branch of the connector, the first branch of the connector extending at an angle to a second branch of the connector, the second including a stopcock.

4. A device for collecting a biological sample as set forth in claim 1 wherein the collection portion includes a first axial end portion and a second axial end portion, the second axial end portion having a collapsed position and an expanded position, the second axial end portion moving in an axial direction relative to the first axial end portion when the second axial end portion moves between the collapsed position and the expanded position, the second axial end portion extending axially into the first axial end portion and having a concave shape when the second axial end portion is in the collapsed position.

5. A device for collecting a biological sample as set forth in claim 1 wherein the second axial end portion has an outer surface facing radially outwardly when the second axial end portion is in the expanded condition, the outer surface facing radially inwardly when the second axial end portion is in the collapsed position.

6. A device for collecting a biological sample as set forth in claim 4 wherein the second axial end portion includes a plurality of tissue collecting projections extending from an outer surface of the second axial end portion.

7. A device for collecting a biological sample as set forth in claim 6 wherein a first side wall of the tissue collecting projection extends generally perpendicular to the outer surface of the second axial end portion and a second wall of the tissue collection projection tapers toward the first side wall as the side walls extend radially outward from the outer surface when the second axial end portion is in a non-inflated position between the collapsed and expanded positions.

8. A device for collecting a biological sample as set forth in claim 7 wherein a lip extends from a radially outer surface of the projection toward the first side wall of the tissue collecting projection when the second axial end portion is in the expanded position.

9. A device for collecting a biological sample as set forth in claim 8 wherein at least one of the tissue collecting projections has a V-shape, the first side wall facing in a proximal direction and forming an inner wall of the V-shape, the second side wall facing in a distal direction and forming an outer wall of the V-shape.

10. A device for collecting a biological sample as set forth in claim 6 wherein the second axial end portion has a durometer between 5-90 Shore A.

11. A device for collecting a biological sample as set forth in claim 6 wherein the second axial end portion has a durometer between 20-70 Shore A.

12. A device for collecting a biological sample as set forth in claim 4 further including a sleeve in the first axial end portion.

13. A device for collecting a biological sample as set forth in claim 10 wherein the sleeve is held in the first axial end portion by an undercut rim on the first axial end portion.

14. A device for collecting a biological sample as set forth in claim 4 wherein the second axial end portion includes a plurality of tissue collecting projections extending from an outer surface of the second axial end portion, each of the projections having a V-shape connected to an adjacent V-shaped projection.

15. A device for collecting a biological sample as set forth in claim 4 further including a cap extending over the second axial end portion when the second axial end portion is in the collapse position to retain the second axial end portion in the collapsed position.

16. A device for collecting a biological sample as set forth in claim 4 further including a weight connected to the first axial end portion.

17. A device for collecting a biological sample in an esophagus of a patient, the device comprising:
a collection portion having a first axial end portion and a second axial end portion, the second axial end portion having a collapsed position and an expanded position, the second axial end portion moving in an axial direction relative to the first axial end portion when the second axial end portion moves between the collapsed position and the expanded position, the second axial end portion extending axially into the first axial end portion and having a concave shape when the second axial end portion is in the collapsed position; and
a sleeve in the first axial end portion.

18. A device for collecting a biological sample as set forth in claim 17 wherein the sleeve is held in the first axial end portion by an undercut rim on the first axial end portion.

19. A device for collecting a biological sample as set forth in claim 17 wherein the second axial end portion extends axially into the sleeve when the second axial end portion is in the collapsed position.

20. A device for collecting a biological sample as set forth in claim 17 wherein the collection portion is swallowable and a stylet is connected with the collection portion for placing the collection portion into the back of a throat of a patient for swallowing.

21. A device for collecting a biological sample as set forth in claim 17 wherein the second axial end portion has an outer surface facing radially outwardly when the second axial end portion is in the expanded condition, the outer surface facing radially inwardly when the second axial end portion is in the collapsed position.

22. A device for collecting a biological sample as set forth in claim 17 wherein the second axial end portion has a durometer between 5-90 Shore A.

23. A device for collecting a biological sample as set forth in claim 17 wherein the second axial end portion has a durometer between 20-70 Shore A.

24. A device for collecting a biological sample in an esophagus of a patient, the device comprising a collection portion having a collapsed position and an expanded position, at least one tissue collecting projection extending from an outer surface of the collection portion, a first side wall of the tissue collecting projection extending generally perpendicular to the outer surface of the collection portion when the collection portion is in a non-inflated position between the collapsed and expanded positions, a second wall of the tissue collecting projection tapering toward the first side wall as the side walls extend radially outward from the outer surface when the collection portion is in the non-inflated position between the collapsed and expanded positions.

25. A device for collecting a biological sample as set forth in claim 24 wherein a lip extends from a radially outer surface of the projection toward the first side wall of the tissue collecting projection when the collection portion is in the expanded position.

26. A device for collecting a biological sample as set forth in claim 25 wherein at least one of the tissue collecting projections has a V-shape, the first side wall facing in a proximal direction and forming an inner wall of the V-shape, the second side wall facing in a distal direction and forming an outer wall of the V-shape.

27. A method for collecting a biological sample from a collection site of an esophagus of a patient, the method comprising:
moving a swallowable collection portion of a device with a stylet into the back of a throat of a patient for swallowing;
moving the collection portion to the collection site in the esophagus with a portion of the collection portion in a collapsed position;
expanding the portion of the collection portion when the collection portion is at the collection site;
collecting a biological sample with the portion of the collection portion in the expanded position;
collapsing the portion of the collection portion after collecting the sample; and
removing the device from the esophagus of the patient.

28. A method for collecting a biological sample from an esophagus as set forth in claim 27 further including moving the collection portion with the portion of the collection portion in the collapsed position in a proximal direction so that the collection portion engages the lower esophageal sphincter (LES) and creates tension in the device, the step of expanding the portion of the collection portion being performed after moving the collection portion into engagement with the LES.

29. A method for collecting a biological sample from an esophagus as set forth in claim 27 wherein the step of expanding the portion of the collection portion includes axially moving a second axial end portion of the collection portion relative to a first axial end portion of the collection portion from a collapsed position into an expanded position and the step of collapsing the portion of the collection portion includes axially moving the second axial end portion into the first axial end portion from the expanded position into the collapsed position.

30. A method for collecting a biological sample from an esophagus as set forth in claim 27 further including providing the collection portion with a plurality of tissue collecting projections extending from an outer surface of the collection portion, each of the tissue collecting projections having a first side wall extending generally perpendicular to the outer surface of the second axial end portion and a second wall tapering toward the first side wall as the side walls extend radially outward from the outer surface when the collection portion is in a non-inflated position between the collapsed and expanded positions.

31. A method for collecting a biological sample from an esophagus as set forth in claim 30 further including forming a lip extending from a radially outer surface of the projection toward the first side wall of the tissue collecting projection when expanding the collection portion.
